(19) United States
(12) Patent Application Publication
(10) Pub. No.: US 2019/0142307 A1
(43) Pub. Date: May 16, 2019

(54) Sensor data management

(71) Applicant: Amer Sports Digital Services Oy, Vantaa (FI)

(72) Inventors: Tuomas Hapola, Vantaa (FI); Mikko Martikka, Vantaa (FI); Timo Eriksson, Vantaa (FI); Erik Lindman, Vantaa (FI)

(21) Appl. No.: 16/228,981

(22) Filed: Dec. 21, 2018

Related U.S. Application Data
(63) Continuation-in-part of application No. 15/382,763, filed on Dec. 19, 2016.
Application No. 15/382,763 is a continuation-in-part of application No. 15/386,050, filed on Dec. 21, 2016.
Application No. 15/386,050 is a continuation-in-part of application No. 15/386,062, filed on Dec. 21, 2016.
Application No. 15/386,062 is a continuation-in-part of application No. 15/386,074, filed on Dec. 21, 2016.

(30) Foreign Application Priority Data
Dec. 21, 2015 (FI) 20155989
Dec. 21, 2015 (GB) 1522525.3
Sep. 20, 2016 (FI) 20165707
Sep. 20, 2016 (FI) 20165709
Sep. 20, 2016 (FI) 20165710

(51) Int. Cl. A61B 5/11; A61B 5/00; G01C 22/00; G01P 15/08; G01P 13/00

(52) U.S. Cl. CPC A61B 5/1123; A61B 5/7267; G01P 13/00; G01C 22/006; G01P 15/0802; A61B 5/0004

(57) ABSTRACT

According to an example aspect of the present invention, there is provided a personal multi-sensor apparatus comprising a memory configured to store plural sequences of sensor data elements and at least one processing core configured to: derive, from the plural sequences of sensor data elements, plural sensor data segments, each sensor data segment comprising time-aligned sensor data element sub-sequences from at least two of the sequences of sensor data elements, and assign a label to at least some of the sensor data segments based on the sensor data elements comprised in the respective sensor data segments, to obtain a sequence of labels

### RELATED APPLICATIONS

This application is a continuation-in-Part of U.S. patent application Ser. No. 15/382,763, filed on Dec. 19, 2016, which claims priority to Finnish patent application No. 20155989, filed on Dec. 21, 2015, Ser. No. 15/386,050, claiming priority of Finnish patent application 20165707, Ser. No. 15/386,062, which claims the priority of Finnish patent application 20165709, and Ser. No. 15/386,074, claiming the priority of Finnish patent application 20165710. The subject matter of these is incorporated by reference in their entirety.

### FIELD

The present invention relates to managing user data generated from sensor devices.

### BACKGROUND

User sessions, such as activity sessions, may be recorded, for example in notebooks, spreadsheets or other suitable media. Recorded training sessions enable more systematic training, and progress toward set goals can be assessed and tracked from the records so produced. Such records may be stored for future reference, for example to assess progress an individual is making as a result of the training. An activity session may comprise a training session or another kind of session.

Personal sensor devices, such as, for example, sensor buttons, smart watches, smartphones or smart jewellery, may be configured to produce sensor data for session records. Such recorded sessions may be useful in managing physical training, child safety or in professional uses. Recorded sessions, or more generally sensor-based activity management, may be of varying type, such as, for example, running, walking, skiing, canoeing, wandering, or assisting the elderly.

Recorded sessions may be viewed using a personal computer, for example, wherein recordings may be copied from a personal device to the personal computer. Files on a personal computer may be protected using passwords and/or encryption, for example.

Personal devices may be furnished with sensors, which may be used, for example, in determining a location, acceleration, or rotation of the personal device. For example, a satellite positioning sensor may receive positioning information from a satellite constellation, and deduce therefrom where the personal device is located. A recorded training session may comprise a route determined by repeatedly determining the location of the personal device during the training session. Such a route may be later observed using a personal computer, for example.

### SUMMARY OF THE INVENTION

The invention is defined by the features of the independent claims. Some specific embodiments are defined in the dependent claims.

According to a first aspect of the present invention, there is provided a personal multi-sensor apparatus comprising a memory configured to store plural sequences of sensor data elements and at least one processing core configured to: derive, from the plural sequences of sensor data elements, plural sensor data segments, each sensor data segment comprising time-aligned sensor data element sub-sequences from at least two of the sequences of sensor data elements, and assign a label to at least some of the sensor data segments based on the sensor data elements comprised in the respective sensor data segments, to obtain a sequence of labels.

According to a second aspect of the present invention, there is provided a method in a personal multisensor apparatus, comprising storing plural sequences of sensor data elements, deriving, from the plural sequences of sensor data elements, plural sensor data segments, each sensor data segment comprising time-aligned sensor data element sub-sequences from at least two of the sequences of sensor data elements, and assigning a label to at least some of the sensor data segments based on the sensor data elements comprised in the respective sensor data segments, to obtain a sequence of labels.

According to a third aspect of the present invention, there is provided a server apparatus comprising a receiver configured to receive a sequence of labels assigned based on sensor data elements, the sensor data elements not being comprised in the sequence of labels, and at least one processing core configured to determine, based on the sequence of labels, an activity type a user has engaged in.

According to a fourth aspect of the present invention, there is provided a method in a server apparatus, comprising receiving a sequence of labels assigned based on sensor data elements, the sensor data elements not being comprised in the sequence of labels, and determining, based on the sequence of labels, an activity type a user has engaged in.

According to a fifth aspect of the present invention, there is provided a non-transitory computer readable medium having stored thereon a set of computer readable instructions that, when executed by at least one processor, cause an apparatus to at least store plural sequences of sensor data elements, derive, from the plural sequences of sensor data elements, plural sensor data segments, each sensor data segment comprising time-aligned sensor data element sub-sequences from at least two of the sequences of sensor data elements, and assign a label to at least some of the sensor data segments based on the sensor data elements comprised in the respective sensor data segments, to obtain a sequence of labels.

According to a sixth aspect of the present invention, there is provided a non-transitory computer readable medium having stored thereon a set of computer readable instructions that, when executed by at least one processor, cause an apparatus to at least receive a sequence of labels assigned based on sensor data elements, the sensor data elements not being comprised in the sequence of labels, and determine, based on the sequence of labels, an activity type a user has engaged in.

According to a seventh aspect of the present invention, there is provided a computer program configured to cause a method in accordance with at least one of the second and fourth aspects to be performed.

### BRIEF DESCRIPTION OF THE DRAWINGS

FIG. 1 illustrates an example system in accordance with at least some embodiments of the present invention;

FIG. 2A illustrates an example multisensorial time series;

FIG. 2B illustrates a second example multisensorial time series;

FIG. 3 illustrates an example apparatus capable of supporting at least some embodiments of the present invention;

FIG. 4 illustrates signalling in accordance with at least some embodiments of the present invention, and

FIG. 5 is a flow graph of a method in accordance with at least some embodiments of the present invention.

### EMBODIMENTS

Sensor data produced in a user device may consume resources in storing or processing it due to its large volume. Consequently, reducing the volume of such sensor data is of interest. Reducing the volume of the sensor data should aim to reduce the sensor data volume while maintaining a usability of the sensor data. Described herein are methods to replace raw sensor data with semantic interpretations of the raw sensor data, in the form of labels assigned to segments of the sensor data, greatly reducing the volume of the data while maintaining its meaning.

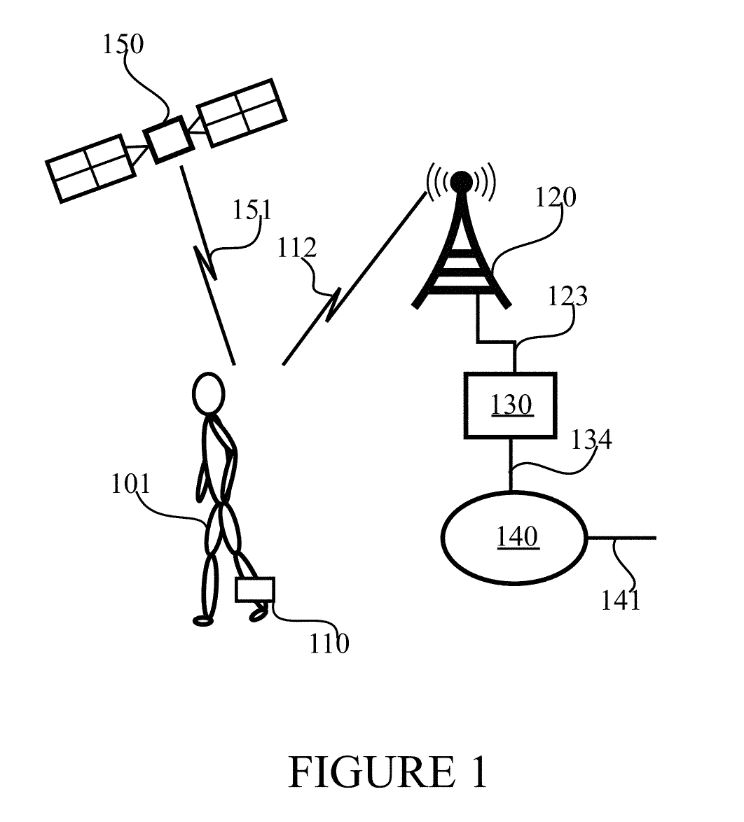

FIG. 1 illustrates an example system in accordance with at least some embodiments of the present invention. The system comprises device 110, which may comprise, for example, multi-sensor device, such as, for example, a personal multi-sensor device, such as, for example, a personal biosensor apparatus such as a smart watch, digital watch, sensor button, or another type of suitable device. In general, a biosensor apparatus may comprise a fitness sensor apparatus or a therapy sensor apparatus, for example. In the illustrated example, device 110 is attached to the user's ankle, but it may equally be otherwise associated with the user, for example by being worn around the wrist. A sensor button is a device comprising a set of sensors and communications interface, configured to produce from each sensor a sequence of sensor data elements. A sensor button may be powered by a battery, or it may gain its energy from movements of the user, for example. The multi-sensor device may comprise an internet of things, IoT, device, for example.

The sensors may be configured to measure acceleration, rotation, moisture, pressure and/or other variables, for example. In one specific embodiment, the sensors are configured to measure acceleration along three mutually orthogonal axes and rotation about three mutually orthogonal axes. The sensors may comprise single- or multi-axis magnetic field sensors, skin signal EMG, ECG, heartbeat and/or optical pulse sensors. Additionally or alternatively, human activity may be sensed via motion or use of sport utensils, tools, machinery and/or devices. In all, such sensors would produce six sequences of sensor data elements, such that in each sequence the sensor data elements are in chronological order, obtained once per sampling interval. The sampling intervals of the sensors do not need to be the same.

Device 110 may be communicatively coupled, directly or indirectly, with a communications network. For example, in FIG. 1 device 110 is coupled, via wireless link 112, with base station 120. Base station 120 may comprise a cellular or non-cellular base station, wherein a non-cellular base station may be referred to as an access point. Examples of cellular technologies include wideband code division multiple access, WCDMA, and long term evolution, LTE, while examples of non-cellular technologies include wireless local area network, WLAN, and worldwide interoperability for microwave access, WiMAX. Base station 120 may be coupled with network node 130 via connection 123. Connection 123 may be a wire-line connection, for example. Network node 130 may comprise, for example, a controller or gateway device. Network node 130 may interface, via connection 134, with network 140, which may comprise, for example, the Internet or a corporate network. Network 140 may be coupled with further networks via connection 141. Network 140 may comprise, or be communicatively coupled, with a back-end server, for example.

Device 110 may be configured to receive, directly or indirectly, from satellite constellation 150, satellite positioning information via satellite link 151. The satellite constellation may comprise, for example the global positioning system, GPS, or the Galileo constellation. Satellite constellation 150 may comprise more than one satellite, although only one satellite is illustrated in FIG. 1 for the same of clarity. Likewise, receiving the positioning information over satellite link 151 may comprise receiving data from more than one satellite.

Where device 110 is indirectly coupled with the communications network and/or satellite constellation 150, it may be arranged to communicate with a personal device of user 101, such as a smartphone, which has connectivity with the communications network and/or satellite constellation 150. Device 110 may communicate with the personal device via, for example, a short-range communication technology such as the Bluetooth or Wibree technologies, or, indeed, via a cable. The personal device and device 110 may be considered to form a personal area network, PAN.

Alternatively or additionally to receiving data from a satellite constellation, device 110 or the personal device may obtain positioning information by interacting with a network in which base station 120 is comprised. For example, cellular networks may employ various ways to position a device, such as trilateration, multilateration or positioning based on an identity of a base station with which attachment is possible or ongoing. Likewise a non-cellular base station, or access point, may know its own location and provide it to device 110 or the personal device, enabling device 110 and/or the personal device to position itself within communication range of this access point. Device 110 or the personal device may be configured to obtain a current time from satellite constellation 150, base station 120 or by requesting it from the user, for example.

Device 110 or the personal device may be configured to provide an activity session. An activity session may be associated with an activity type. Examples of activity types include rowing, paddling, cycling, jogging, walking, hunting, swimming and paragliding. In a simple form, an activity session may comprise storing sensor data produced with sensors comprised in device 110, the personal device or a server, for example. An activity session may be determined to have started and ended at certain points in time, such that the determination takes place afterward or concurrently with the starting and/or ending. In other words, device 110 may store sensor data to enable subsequent identification of activity sessions based at least partly on the stored sensor data.

An activity session may enhance a utility a user can obtain from the activity, for example, where the activity involves movement outdoors, the activity session may provide a recording of the activity session. A recording of an activity session may, in some embodiments, provide the user with contextual information. Such contextual information may comprise, for example, locally relevant weather information, received via base station 120, for example. Such contextual information may comprise at least one of the following: a rain warning, a temperature warning, an indication of time remaining before sunset, an indication of a nearby service that is relevant to the activity, a security warning, an indication of nearby users and an indication of a nearby location where several other users have taken photographs. Contextual information may be presented during an activity session.

A recording of an activity session may comprise information on at least one of the following: a route taken during the activity session, a metabolic rate or metabolic effect of the activity session, a time the activity session lasted, a quantity of energy consumed during the activity session, a sound recording obtained during the activity session and an elevation map along the length of the route taken during the activity session. A route may be determined based on positioning information, for example. Metabolic effect and consumed energy may be determined, at least partly, based on sensor data obtained from user 101 during the activity session. A recording may be stored in device 110, the personal device, or in a server or other cloud data storage service. A recording stored in a server or cloud may be encrypted prior to transmission to the server or cloud, to protect privacy of the user. A recording may be produced even if the user has not indicated an activity session has started, since a beginning and ending of an activity session may be determined after the session has ended, for example based, at least partly, on sensor data.

After an activity has ended, device 110 may have stored therein, or in a memory to which device 110 has access, plural sequences of sensor data elements. The stored sequences of sensor data elements may be stored in chronological order as a time series that spans the activity session as well as time preceding and/or succeeding the activity session. The beginning and ending points in time of the activity session may be selected from the time series by the user, or dynamically by device 110. For example, where, in the time series, acceleration sensor data begins to indicate more active movements of device 110, a beginning point of an activity session may be selected. Such a change may correspond to a time in the time series when the user stopped driving a car and began jogging, for example. Likewise, a phase in the time series where the more active movements end may be selected as an ending point of the activity session.

As described above, the plural sequences of sensor data elements may comprise data from more than one sensor, wherein the more than one sensor may comprise sensors of at least two distinct types. For example, plural sequences of sensor data elements may comprise sequences of acceleration sensor data elements and rotation sensor data elements. Further examples are sound volume sensor data, moisture sensor data and electromagnetic sensor data. In general, each sequence of sensor data elements may comprise data from one and only one sensor.

An activity type may be determined based, at least partly, on the sensor data elements. This determining may take place when the activity is occurring, or afterwards, when analysing the sensor data. The activity type may be determined by device 110 or by a server-side computer that has access to the sensor data, for example, or a server that is provided access to the sensor data. Where a server is given access to the sensor data, or, in some embodiments, when activity type detection is performed on device 110 or the personal device, the sensor data may be processed into a sequence of labels.

A sequence of labels may characterize the content of sensor data. For example, where the sensor data elements are numerical values obtained during jogging, a sequence of labels derived from those sensor data elements may comprise a sequence of labels: {jog-step, jog-step, jog-step, jog-step, jog-step, . . . }. Likewise, where the sensor data elements are numerical values obtained during a long jump, a sequence of labels derived from those sensor data elements may comprise a sequence of labels: {sprint-step, sprint-step, sprint-step, sprint-step, sprint-step, leap, stop}. Likewise, where the sensor data elements are numerical values obtained during a triple jump, a sequence of labels derived from those sensor data elements may comprise a sequence of labels: {sprint-step, sprint-step, sprint-step, sprint-step, leap, leap, leap, stop}. The sequences of labels are thus usable in identifying the activity type, for example differentiating between long jump and triple jump based on the number of leaps.

The labels may be expressed in natural language or as indices to a pre-defined table, which may be dynamically updatable, as new kinds of exercise primitives become known. For example, in the table a jog-step may be represented as 01, a sprint-step (that is, a step in running much faster than jogging) as 02, a leap as 03, and a stopping of motion may be represented as 04. Thus the triple jump would be represented as a sequence of labels {02, 02, 02, 02, 03, 03, 03, 04}. The activity, for example a triple jump, may be detected from the labels, while the sequence of labels takes up significantly less space than the original sequences of sensor data elements.

To process the sequences of sensor data elements into a sequence of labels, sensor data segments may be derived from the sequences of sensor data elements. Each sensor data segment may then be associated with an exercise primitive and assigned a label, to obtain the sequence of labels. Each sensor data segment may comprise time-aligned sensor data element sub-sequences from at least two of the sequences of sensor data elements. In other words, segments of sensor data are derived, each such segment comprising a time slice of original sequences of sensor data elements. This may be conceptualized as time-slicing a multi-sensor data stream captured during jogging into the individual steps that make up the jogging session. Likewise other activity sessions may be time-sliced into exercise primitives which make up the activity.

To derive the segments, device 110 or another device may be configured to analyse the sequences of sensor data elements to identify therein units. Each segment may comprise slices of the sequences of sensor data elements, the slices being time-aligned, that is, obtained at the same time from the respective sensors.

For example, steps in running are repetitive in nature, wherefore identifying a pattern in the sequences of sensor data elements which repeats at a certain frequency is a clue the sequences may be segmented according to this frequency. A frequency may be identified, for example, by performing a fast fourier transform, FFT, on each of the sequences of sensor data elements, and then averaging the resulting spectrum, to obtain an overall frequency characteristic of the sequences of sensor data elements.

In case of motion, one way to segment the sensor data is to try to construct a relative trajectory of the sensor device. One way to estimate this trajectory is to double integrate the x-, y-, and z-components of acceleration sensor outputs. In this process one may remove gravity induced biases. Mathematically this can be done by calculating the baseline of each output. One way is to filter the data as in the next equation.

acc_i_baseline=acc_i_baseline+coeff_a*(acc_i−acc_i_baseline)

Acc above refers to the acceleration measurement and i refers to its components x, y, and z. These filtered values can be subtracted from the actual measurements: acc_i_without G=acc_i_−acc_i_baseline. This is a rough estimate of the true linear acceleration, but still a fast and robust way to estimate it. The integration of these linear acceleration values leads to the estimate of the velocity of the sensor device in three-dimensional, 3D, space. The velocity components have biases due the incomplete linear acceleration estimate. These biases may be removed like in the previous equation:

v_i_baseline=v_i_baseline+coeff_v*(v_i−v_i_baseline)

V above refers to the velocity estimate and I refers to its components x, y, and z. These velocity components are not true velocities of the sensor device, but easily and robustly calculated estimates of them. The baseline components may be subtracted from the velocity estimates before integration: v_i_wo_bias=v_i−v_i_baseline. Since the method so far is incomplete, the integrals of the velocity components produce biased position estimates p_x, p_y, and p_z. Therefore these biases needs to be removed like in the previous equations:

p_i_baseline=p_i_baseline+coeff_p*(p_i−p_i_baseline)

P above refers to the position estimate and i refers to its components. Since this procedure effectively produces 0-mean values, the natural reference of position is p_x_ref=0, p_y_ref=0, and p_z_ref=0. The Euclidean distances of the measured values sqrt(p_x_ti**2+p_y_ti**2+p_z_ti**2) form a time series varying from 0 to some maximum value. ti refers to the index in the time series. These maximum values can detected easily. The moment in time of the maximum value starts and the next maximum value end the segment (and starts the next segment). The detection of the maximum value can be conditional i.e. the maximum value is accepted as a start/stop marker only when it exceeds a certain level.

Also, the above described procedure to calculate the relative trajectory can be more precise by utilizing the gyroscopes and using e.g. complementary filtering.

Other ways to segment the data, that is, derive the segments, may include fitting to a periodic model, using a suitably trained artificial neural network or using a separate segmenting signal provided over a radio or wire-line interface, for example. The segmenting signal may be correlated in time with the sequences of sensor data elements, to obtain the segments. A segmenting signal may be transmitted or provided by a video recognition system or pressure pad system, for example. Such a video recognition system may be configured to identify steps, for example.

Once the segments have been derived, each segment may be assigned a label. Assigning the label may comprise identifying the segment. The identification may comprise comparing the sensor data comprised in the segment to a library of reference segments, for example in a least-squares sense, and selecting from the library of reference segments a reference segment which most resembles the segment to be labelled. The label assigned to the segment will then be a label associated with the closest reference segment in the library of reference segments.

In some embodiments, a plurality of reference segment libraries is used, such that a first phase of the identification is selection of a reference segment library. For example, where two reference segment libraries are used, one of them could be used for continuous activity types and a second one of them could be used for discontinuous activity types. The continuous activity type is selected where the sequences of sensor data elements reflect a repetitive action which repeats a great number of times, such as jogging, walking, cycling or rowing. The discontinuous activity type is selected when the activity is characterized by brief sequences of action which are separated from each other in time, for example the afore-mentioned triple jump, or pole vault, being examples. Once the reference segment library is chosen, all the segments are labelled with labels from the selected reference segment library.

A benefit of first selecting a reference segment library is obtained in more effective labelling, as there is a lower risk segments are assigned incorrect labels. This is so, since the number of reference segments the sensor data segments are compared to is lower, increasing the chances a correct one is chosen.

Once the segments have been labelled, a syntax check may be made wherein it is assessed, if the sequence of labels makes sense. For example, if the sequence of labels is consistent with known activity types, the syntax check is passed. On the other hand, if the sequence of labels comprises labels which do not fit together, a syntax error may be generated. As an example, a sequence of jogging steps which comprises mixed therein a few paddling motions would generate a syntax error, since the user cannot really be jogging and paddling at the same time. In some embodiments, a syntax error may be resolved by removing from the sequence of labels the labels which do not fit in, in case they occur in the sequence of labels only rarely for example at a rate of less than 2%.

The reference segment libraries may comprise indications as to which labels fit together, to enable handling syntax error situations.

Different exercise primitives may be associated with different characteristic frequencies. For example, acceleration sensor data may reflect a higher characteristic frequency when the user has been running, as opposed to walking. Thus the labelling of the segments may be based, in some embodiments, at least partly, on deciding which reference segment has a characteristic frequency that most closely matches a characteristic frequency of a section of the sequence of sensor data elements under investigation. Alternatively or in addition, acceleration sensor data may be employed to determine a characteristic movement amplitude.

The reference segment libraries may comprise reference datasets that are multi-sensorial in nature in such a way, that each reference segment comprises data that may be compared to each sensor data type that is available. For example, where device 110 is configured to compile a time series of acceleration and sound sensor data types, the reference segments may comprise reference datasets, each reference segment corresponding to a label, wherein each reference segment comprises data that may be compared with the acceleration data and data that may be compared with the sound data, for example. The determined label may be determined as the label that is associated with the multi-sensorial reference segment that most closely matches the segment stored by device 110, for example. Device 110 may comprise, for example, microphones and cameras. Furthermore a radio receiver may, in some cases, be configurable to measure electric or magnetic field properties. Device 110 may comprise a radio receiver, in general, where device 110 is furnished with a wireless communication capability.

An example of activity type identification by segmenting and labelling is swimming, wherein device 110 stores sequences of sensor data elements that comprise moisture sensor data elements and magnetic field sensor data elements. The moisture sensor data elements indicating presence of water would cause a water-sport reference segment library to be used. Swimming may involve elliptical movements of an arm, to which device 110 may be attached, which may be detectable as periodically varying magnetic field data. In other words, the direction of the Earth's magnetic field may vary from the point of view of the magnetic field sensor in a periodic way in the time series. This would enable labelling the segments as, for example, breast-stroke swimming motions.

Overall, a determined, or derived, activity type may be considered an estimated activity type until the user has confirmed the determination is correct. In some embodiments, a few, for example two or three, most likely activity types may be presented to the user as estimated activity types for the user to choose the correct activity type from. Using two or more types of sensor data increases a likelihood the estimated activity type is correct. Once the user confirms or selects a specific activity type, labelling of segments may be enforced to be compliant with this activity type. This may mean, for example, that the set of reference segments the sensor data segments are compared to is limited to reference data segments consistent with this activity type.

Where device 110 or a personal device assigns the labels, the sequence of labels may be transmitted to a network server, for example, for storage. Device 110, the personal device or the server may determine an overall activity type the user is engaged in, based on the labels. This may be based on a library of reference label sequences, for example.

In general, device 110 or the personal device may receive a machine readable instruction, such as an executable program or executable script, from the server or another network entity. The machine readable instruction may be usable in determining activity type from the sequence of labels, and/or in assigning the labels to sensor data segments. In the latter case, the machine readable instruction may be referred to as a labelling instruction.

The process may adaptively learn, based on the machine readable instructions, how to more accurately assign labels and/or determine activity types. A server may have access to information from a plurality of users, and high processing capability, and thus be more advantageously placed to update the machine-readable instructions than device 110, for example.

The machine readable instructions may be adapted by the server. For example, a user who first obtains a device 110 may initially be provided, responsive to messages sent from device 110, with machine readable instructions that reflect an average user population. Thereafter, as the user engages in activity sessions, the machine readable instructions may be adapted to more accurately reflect use by this particular user. For example, limb length may affect periodical properties of sensor data captured while the user is swimming or running. To enable the adapting, the server may request sensor data from device 110, for example periodically, and compare sensor data so obtained to the machine readable instructions, to hone the instructions for future use with this particular user. Thus a beneficial effect is obtained in fewer incorrectly labelled segments, and more effective and accurate compression of the sensor data.

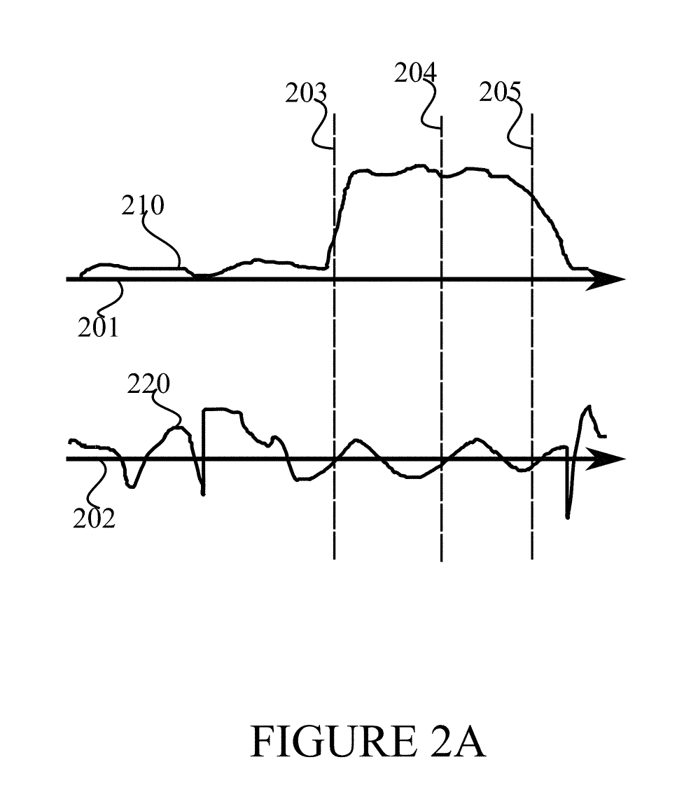

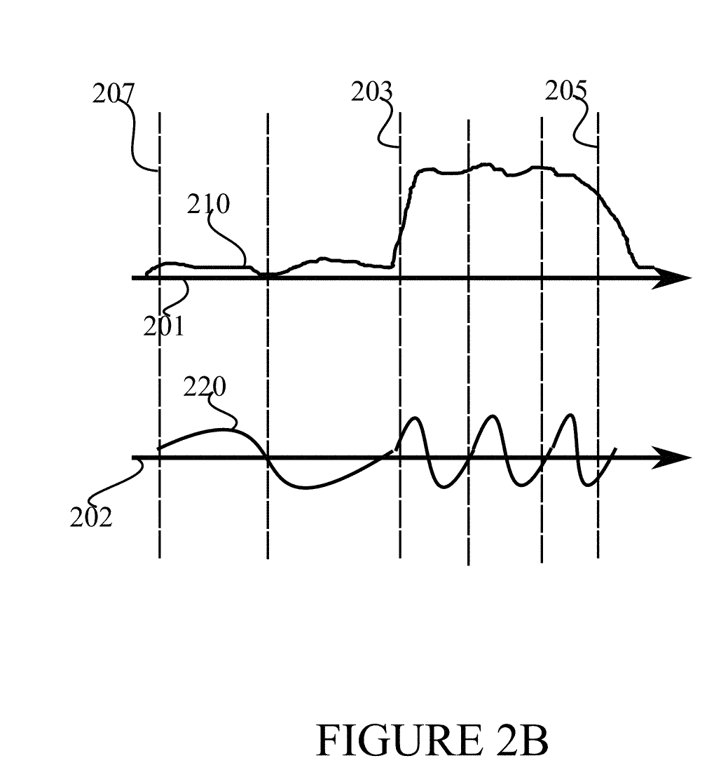

FIG. 2A illustrates an example of plural sequences of sensor data elements. On the upper axis, 201, is illustrated a sequence of moisture sensor data elements 210 while the lower axis, 202, illustrates a time series 220 of deviation of magnetic north from an axis of device 110, that is, a sequence of magnetic sensor data elements.

The moisture sequence 210 displays an initial portion of low moisture, followed by a rapid increase of moisture that then remains at a relatively constant, elevated, level before beginning to decline, at a lower rate than the increase, as device 110 dries.

Magnetic deviation sequence 220 displays an initial, erratic sequence of deviation changes owing to movement of the user as he operates a locker room lock, for example, followed by a period of approximately periodic movements, before an erratic sequence begins once more. The wavelength of the periodically repeating motion has been exaggerated in FIG. 2 to render the illustration clearer.

A swimming activity type may be determined as an estimated activity type, beginning from point 203 and ending in point 205 of the sequences. In detail, the sequences may be segmented into two segments, firstly from point 203 to point 204, and secondly from point 204 to point 205. As the moisture sensor indicates water sports, a water sports reference segment library is used to label the segments as, for example, freestroke swimming segments. The sequence of labels would thus be {freestroke, freestroke}. Of course, in actual swimming the number of segment would be much higher, but two segments are illustrated in FIG. 2 for the sake of simplicity. Overall, the two sensor data segments, from 203 to 204 and from 204 to 205, both comprise time-aligned sensor data element sub-sequences from sequences 210 and 220.

FIG. 2B illustrates a second example of plural sequences of sensor data elements. In FIG. 2B, like numbering denotes like elements as in FIG. 2A. Unlike in FIG. 2A, not one but two activity sessions are determined in the time series of FIG. 2B. Namely, a cycling session is determined to start at beginning point 207 and to end at point 203, when the swimming session begins. Thus the compound activity session may relate to triathlon, for example. In cycling, moisture remains low, and magnetic deviation changes only slowly, for example as the user cycles in a velodrome. The segments would thus comprise two segments between points 207 and 203, and three segments between points 203 and 205. The sequence of labels could be {cycling, cycling, freestroke, freestroke, freestroke}. Again, the number of segments is dramatically reduced for the sake of clarity of illustration.

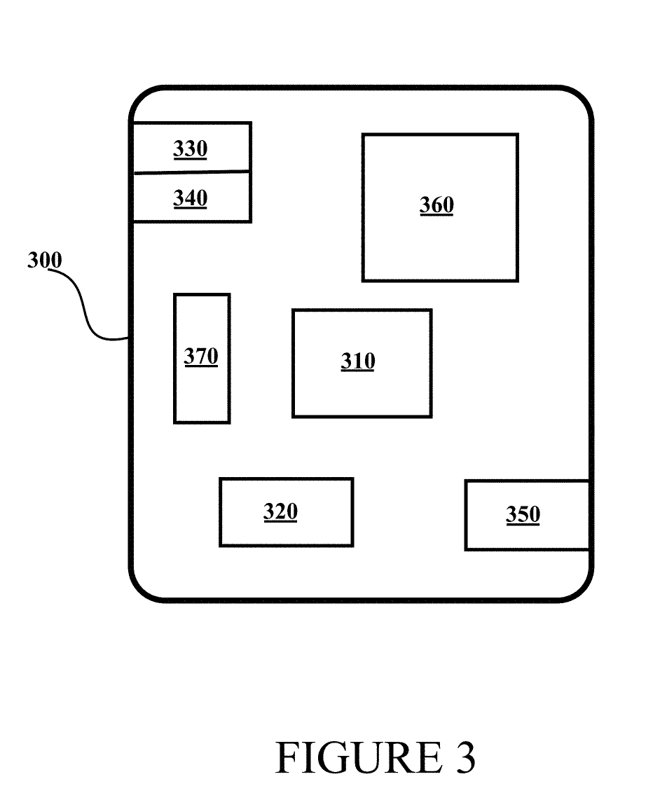

FIG. 3 illustrates an example apparatus capable of supporting at least some embodiments of the present invention. Illustrated is device 300, which may comprise, for example, device 110 of FIG. 1. Comprised in device 300 is processor 310, which may comprise, for example, a single- or multi-core processor wherein a single-core processor comprises one processing core and a multi-core processor comprises more than one processing core. Processor 310 may comprise more than one processor. A processing core may comprise, for example, a Cortex-A8 processing core designed by ARM Holdings or an Excavator processing core produced by Advanced Micro Devices Corporation. Processor 310 may comprise at least one Qualcomm Snapdragon and/or Intel Atom processor. Processor 310 may comprise at least one application-specific integrated circuit, ASIC. Processor 310 may comprise at least one field-programmable gate array, FPGA. Processor 310 may be means for performing method steps in device 300. Processor 310 may be configured, at least in part by computer instructions, to perform actions.

Device 300 may comprise memory 320. Memory 320 may comprise random-access memory and/or permanent memory. Memory 320 may comprise at least one RAM chip. Memory 320 may comprise solid-state, magnetic, optical and/or holographic memory, for example. Memory 320 may be at least in part accessible to processor 310. Memory 320 may be at least in part comprised in processor 310. Memory 320 may be means for storing information. Memory 320 may comprise computer instructions that processor 310 is configured to execute. When computer instructions configured to cause processor 310 to perform certain actions are stored in memory 320, and device 300 overall is configured to run under the direction of processor 310 using computer instructions from memory 320, processor 310 and/or its at least one processing core may be considered to be configured to perform said certain actions. Memory 320 may be at least in part comprised in processor 310. Memory 320 may be at least in part external to device 300 but accessible to device 300.

Device 300 may comprise a transmitter 330. Device 300 may comprise a receiver 340. Transmitter 330 and receiver 340 may be configured to transmit and receive, respectively, information in accordance with at least one cellular or non-cellular standard. Transmitter 330 may comprise more than one transmitter. Receiver 340 may comprise more than one receiver. Transmitter 330 and/or receiver 340 may be configured to operate in accordance with global system for mobile communication, GSM, wideband code division multiple access, WCDMA, long term evolution, LTE, IS-95, wireless local area network, WLAN, Ethernet and/or worldwide interoperability for microwave access, WiMAX, standards, for example.

Device 300 may comprise a near-field communication, NFC, transceiver 350. NFC transceiver 350 may support at least one NFC technology, such as NFC, Bluetooth, Wibree or similar technologies.

Device 300 may comprise user interface, UI, 360. UI 360 may comprise at least one of a display, a keyboard, a touchscreen, a vibrator arranged to signal to a user by causing device 300 to vibrate, a speaker and a microphone. A user may be able to operate device 300 via UI 360, for example to manage activity sessions.

Device 300 may comprise or be arranged to accept a user identity module 370. User identity module 370 may comprise, for example, a subscriber identity module, SIM, card installable in device 300. A user identity module 370 may comprise information identifying a subscription of a user of device 300. A user identity module 370 may comprise cryptographic information usable to verify the identity of a user of device 300 and/or to facilitate encryption of communicated information and billing of the user of device 300 for communication effected via device 300.

Processor 310 may be furnished with a transmitter arranged to output information from processor 310, via electrical leads internal to device 300, to other devices comprised in device 300. Such a transmitter may comprise a serial bus transmitter arranged to, for example, output information via at least one electrical lead to memory 320 for storage therein. Alternatively to a serial bus, the transmitter may comprise a parallel bus transmitter. Likewise processor 310 may comprise a receiver arranged to receive information in processor 310, via electrical leads internal to device 300, from other devices comprised in device 300. Such a receiver may comprise a serial bus receiver arranged to, for example, receive information via at least one electrical lead from receiver 340 for processing in processor 310. Alternatively to a serial bus, the receiver may comprise a parallel bus receiver.

Device 300 may comprise further devices not illustrated in FIG. 3. For example, where device 300 comprises a smartphone, it may comprise at least one digital camera. Some devices 300 may comprise a back-facing camera and a front-facing camera, wherein the back-facing camera may be intended for digital photography and the front-facing camera for video telephony. Device 300 may comprise a fingerprint sensor arranged to authenticate, at least in part, a user of device 300. In some embodiments, device 300 lacks at least one device described above. For example, some devices 300 may lack a NFC transceiver 350 and/or user identity module 370.

Processor 310, memory 320, transmitter 330, receiver 340, NFC transceiver 350, UI 360 and/or user identity module 370 may be interconnected by electrical leads internal to device 300 in a multitude of different ways. For example, each of the aforementioned devices may be separately connected to a master bus internal to device 300, to allow for the devices to exchange information. However, as the skilled person will appreciate, this is only one example and depending on the embodiment various ways of interconnecting at least two of the aforementioned devices may be selected without departing from the scope of the present invention.

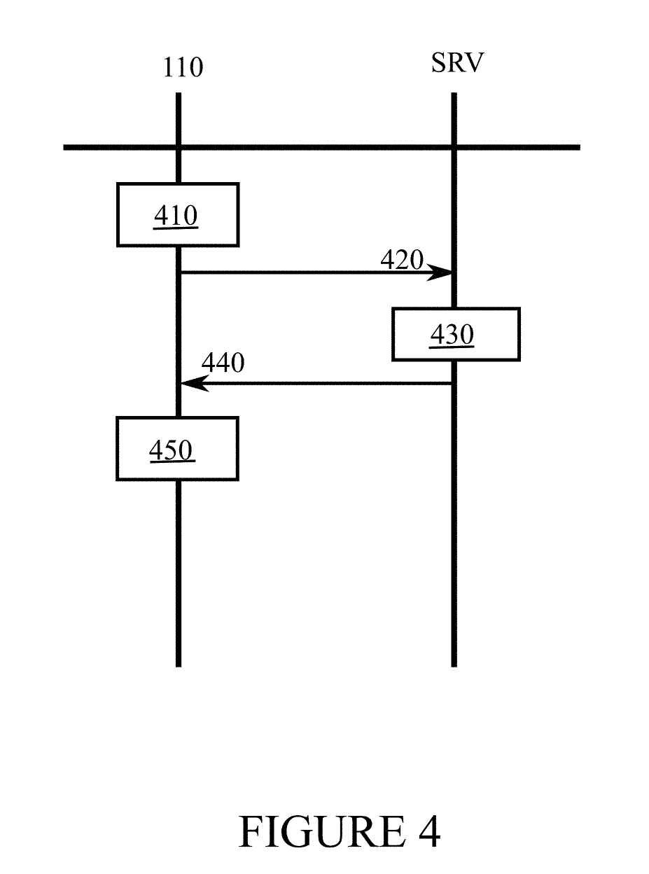

FIG. 4 illustrates signalling in accordance with at least some embodiments of the present invention. On the vertical axes are disposed, on the left, device 110 of FIG. 1, and on the right, a server SRV. Time advances from the top toward the bottom. Initially, in phase 410, device 110 obtains sensor data from at least one, and in some embodiments from at least two sensors. The sensor data may comprise sequences of sensor data elements, as described herein above. The sensor or sensors may be comprised in device 110, for example. The sensor data may be stored in a time series, for example at a sampling frequency of 1 Hz, 10 Hz, 1 Khz or indeed another sampling interval. The sampling interval need not be the same in the various sequences of sensor data elements.

Phase 410 may comprise one or more activity sessions of at least one activity type. Where multiple activity sessions are present, they may be of the same activity type or different activity types. The user need not, in at least some embodiments, indicate to device 110 that activity sessions are ongoing. During phase 410, device 110 may, but in some embodiments need not, identify activity types or sessions. The sequences of sensor data elements compiled during phase 410 may last 10 minutes or 2 hours, for example. As a specific example, the time series may last from the previous time sensor data was downloaded from device 110 to another device, such as, for example, personal computer PC1.

Further, in phase 410, device 110 segments the sequences of sensor data elements to plural sensor data segments, as described herein above. These segments are then assigned labels to obtain a conversion of the sequences of sensor data elements to a sequence of labels.

In phase 420, the sequence of labels is provided, at least partly, to server SRV. This phase may further comprise providing to server SRV optional activity and/or event reference data. The providing may proceed via base station 120, for example. The sequence of labels may be encrypted en route to the server to protect the user's privacy.

In phase 430, server SRV may determine, based at least partly on the sequence of labels in the message of phase 420, an associated machine readable instruction. The machine readable instruction may relate, for example, to improved labelling of segments relating to activities related to the labels in the sequence of labels received in server SRV from device 110 in phase 420.

In phase 440 the machine readable instruction determined in phase 430 is provided to device 110, enabling, in phase 450, a more accurate labelling of segments of sensor data.

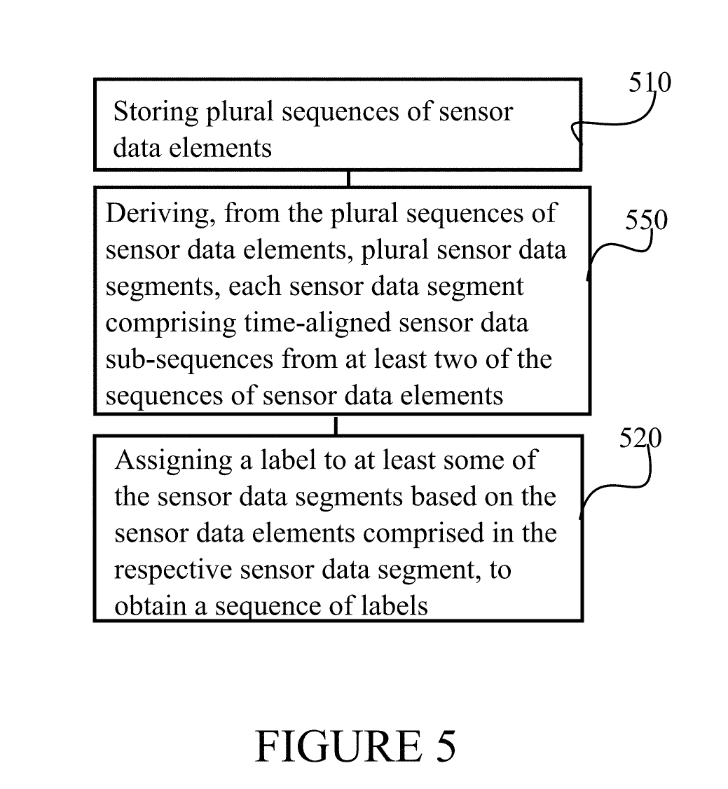

FIG. 5 is a flow graph of a method in accordance with at least some embodiments of the present invention. The phases of the illustrated method may be performed in device 110, an auxiliary device or a personal computer, for example, or in a control device configured to control the functioning thereof, when implanted therein.

Phase 510 comprises storing plural sequences of sensor data elements. Phase 520 comprises deriving, from the plural sequences of sensor data elements, plural sensor data segments, each sensor data segment comprising time-aligned sensor data element sub-sequences from at least two of the sequences of sensor data elements. Finally, phase 530 comprises assigning a label to at least some of the sensor data segment based on the sensor data elements comprised in the respective sensor data segment, to obtain a sequence of labels.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the preceding description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in facilitating analysis of sensor data.

### ACRONYMS LIST

### GPS Global Positioning System

### LTE Long Term Evolution

### NFC Near-Field Communication

### WCDMA Wideband Code Division Multiple Access

WiMAX worldwide interoperability for microwave access
WLAN Wireless local area network

### REFERENCE SIGNS LIST

| | |
|---|---|
| 110 | Device |
| 120 | Base Station |
| 130 | Network Node |
| 140 | Network |
| 150 | Satellite Constellation |
| 201, 202 | Axes in FIG. 2 |
| 203, 2 05, 207 | Activity session endpoints in FIG. 2 and FIG. 2B |
| 210, 220 | Sensor data time series in FIGS. 2 and 2B |
| 310-370 | Structure illustrated in FIG. 3 |
| 410-430 | Phases of the method of FIG. 4 |
| 510-530 | Phases of the method of FIG. 5 |

1. A personal multi-sensor apparatus comprising:
a memory configured to store plural sequences of sensor data elements, and
at least one processing core configured to:
derive, from the plural sequences of sensor data elements, plural sensor data segments, each sensor data segment comprising time-aligned sensor data element sub-sequences from at least two of the sequences of sensor data elements, and
assign a label to at least some of the sensor data segments based on the sensor data elements comprised in the respective sensor data segments, to obtain a sequence of labels.

2. The apparatus according to claim 1, wherein the apparatus is further configured to transmit the sequence of labels to a node in network.

3. The apparatus according to claim 1, wherein the apparatus is further configured to determine, based on the sequence of labels, an activity type a user has engaged in while the sequences of sensor data have been obtained.

4. The apparatus according to claim 3, wherein the apparatus is configured to receive, from a node in a network, a machine readable instruction, and to employ the machine readable instruction in determining the activity type.

5. The apparatus according to claim 4, wherein the machine readable instruction comprises at least one of the following: an executable program and an executable script.

6. The apparatus according to claim 1, wherein the apparatus is configured to receive, from a network, at least one labelling instruction, and to employ the at least one machine readable labelling instruction in the assigning of the label to each sensor data segment.

7. The apparatus according to claim 6, wherein the machine readable labelling instruction comprises at least one of the following: an executable program and an executable script.

8. The apparatus according to claim 1, wherein each of the plural sequences of sensor data elements comprises sensor data elements originating in exactly one sensor.

9. The apparatus according to claim 1, wherein the plural sequences of sensor data elements comprise at least three sequences of sensor data elements.

10. The apparatus according to claim 1, wherein the plural sequences of sensor data elements comprise at least nine sequences of sensor data elements.

11. The apparatus according to claim 1, wherein the apparatus is configured to derive the plural sensor data segments using, at least in part, a suitably trained artificial neural network.

12. A method in a personal multisensor apparatus, comprising:
storing plural sequences of sensor data elements;
deriving, from the plural sequences of sensor data elements, plural sensor data segments, each sensor data segment comprising time-aligned sensor data element sub-sequences from at least two of the sequences of sensor data elements, and
assigning a label to at least some of the sensor data segments based on the sensor data elements comprised in the respective sensor data segments, to obtain a sequence of labels.

13. The method according to claim 12, further comprising transmitting the sequence of labels to a node in network.

14. The method according to claim 12, further comprising determining, based on the sequence of labels, an activity type a user has engaged in while the sequences of sensor data have been obtained.

15. The method according to claim 14, further comprising receiving, from a node in a network, a machine readable instruction, and employing the machine readable instruction in determining the activity type.

16. The method according to claim 15, wherein the machine readable instruction comprises at least one of the following: an executable program and an executable script.

17. The method according to claim 12, further comprising receiving, from a network, at least one labelling instruction, and employing the at least one machine readable labelling instruction in the assigning of the label to each sensor data segment.

18. The method according to claim 17, wherein the machine readable labelling instruction comprises at least one of the following: an executable program and an executable script.

19. A server apparatus comprising:
a receiver configured to receive a sequence of labels assigned based on sensor data elements, the sensor data elements not being comprised in the sequence of labels, and
at least one processing core configured to:
determine, based on the sequence of labels, an activity type a user has engaged in.

20. The server apparatus according to claim 19, wherein the server apparatus is configured to determine the activity type based on comparing the received sequence of labels with a list of label sequences stored in the server apparatus, and by selecting an activity type which is associated with a sequence of labels in the list which matches the received sequence of labels.

21. A method in a server apparatus, comprising:
receiving a sequence of labels assigned based on sensor data elements, the sensor data elements not being comprised in the sequence of labels, and
determining, based on the sequence of labels, an activity type a user has engaged in.

22. The method according to claim 21, wherein the determining of the activity type is based on comparing the received sequence of labels with a list of label sequences stored in the server apparatus, and on selecting an activity type which is associated with a sequence of labels in the list which matches the received sequence of labels.

23. A non-transitory computer readable medium having stored thereon a set of computer readable instructions that, when executed by at least one processor, cause an apparatus to at least:
store plural sequences of sensor data elements;
derive, from the plural sequences of sensor data elements, plural sensor data segments, each sensor data segment comprising time-aligned sensor data element sub-sequences from at least two of the sequences of sensor data elements, and
assign a label to at least some of the sensor data segments based on the sensor data elements comprised in the respective sensor data segments, to obtain a sequence of labels.

24. A non-transitory computer readable medium having stored thereon a set of computer readable instructions that, when executed by at least one processor, cause an apparatus to at least:
receive a sequence of labels assigned based on sensor data elements, the sensor data elements not being comprised in the sequence of labels, and
determine, based on the sequence of labels, an activity type a user has engaged in.

25. (canceled)
